(19) United States
(12) Patent Application Publication
(10) Pub. No.: US 2013/0243891 A1
(43) Pub. Date: Sep. 19, 2013

(54) USES OF NORTH AMERICAN GINSENG FRACTIONS FOR TREATING LEUKEMIA

(71) Applicants: Jacqueline Shan, Edmonton (CA); Sandra Miller, St. Lambert (CA)

(72) Inventors: Jacqueline Shan, Edmonton (CA); Sandra Miller, St. Lambert (CA)

(73) Assignee: fx Life Sciences AG, Zug (CH)

(21) Appl. No.: 13/886,610

(22) Filed: May 3, 2013

Related U.S. Application Data
(63) Continuation of application No. 12/600,301, filed on May 12, 2010, now abandoned.

(51) Int. Cl. A61K 36/258

(52) U.S. Cl. CPC A61K 36/258
USPC 424/728

(57) ABSTRACT

The invention is directed to a method of treating hematological malignancies by administering to the subject ginseng fractions from North American ginseng (Panax quinquefolium). The present invention can be used to activate the proliferation of hemopoietic cells in a patient in need of treatment, or as a therapeutic targeted at conditions characterized by hematological malignancy, such as an abnormal proliferation of blood cells, lymphocytes, multiple myeloma, etc. The invention also includes a method of treating leukemia by activating the proliferation of natural killer (NK) cells and monocytes in the bone marrow and the spleen of the patient by administering at least one ginseng fraction selected from the group consisting of CVT-E002, PQ.sub.2, PQ.sub.223 and purified fractions from CVT-E002, PQ.sub.2 and PQ.sub.223. The present invention may be used to treat the hematological malignancy or as a supplement for patients undergoing chemotherapy or radiation therapy.

### FIELD OF THE INVENTION

This invention relates to a method of treating a hematological malignancy in a patient by administering to the patient effective amounts of fractions made from North American ginseng (Panax quinquefolius). The present invention can be used to activate the proliferation of hemopoietic cells in a patient in need of treatment, or as a therapeutic targeted at conditions characterized by hematological malignancy, such as an abnormal proliferation of blood cells, lymphocytes, multiple myeloma, etc. The present invention may be used to treat the hematological malignancy or as a supplement for patients undergoing chemotherapy or radiation therapy.

### BACKGROUND OF THE INVENTION

For hundreds of years, the use of certain non-toxic agents such as herbal compounds has been widely accepted for a variety of physiological conditions, especially in the Orient. Panax ginseng C. A. Meyer is the best known traditional Chinese medicine. The important pharmacological activities of ginseng extracts, alone or in combination with other drugs, include alleviation of renal impairment, prevention of stress, modulation of immunological responsiveness and inhibition of carcinogenesis. American ginseng, Panax quinquefolius, is another species of ginseng which has gained popularity as a health supplement having many beneficial health effects. Several groups of scientists have attempted to isolate and elucidate the structure of various components present in ginseng to test for the effectiveness of these compounds in treating hematological malignancies. These investigations, unfortunately, have been limited to in vitro studies, as opposed to in vivo studies, in which ginseng-derived compounds have been assayed for their ability to decrease the growth of leukemic tissue cell lines.

Fujimoto et al., in Chem Pharm Bull (Tokyo)., 39(2):521-3 (1991), isolated three cytotoxic polyacetylenes, PQ-1 (1), PQ-2 (2) and PQ-3 (3), from Panax quinquefolius. The structures of these acetylenes were determined by analyses of their 1 H-1 H and 1H-13C COSY spectra. According to Fujimoto et al., these compounds exhibited strong cytotoxic activities against leukemia cells (L 1210) in tissue culture.

Yi et al., in Zhongguo Zhong Xi Yi Jie He Za Zhi, 13(12):722-4, 708 (1993), demonstrated that ginsenosides extracted from stem and leaf of Panax ginseng (GSL) had an inductive differentiation effect on all types of acute nonlymphocytic leukemia cells in primary culture. The effect on M5, M4 was most potent, followed by M1, M2 and the least, on M3. These investigators concluded that the inductive differentiation effect of ginsenosides may be due to the comprehensive effect of increasing intracellular cAMP and inducing interferon.

Hasegawa et al., in Planta Med., 61(5):409-13 (1995), examined the effects of some triterpenoids from Panax (Araliaceae) and Glycyrrhiza (Leguminosae) spp. on the sensitivity to daunomycin (DAU) and vinblastine (VBL) of adriamycin (ADM)-resistant P388 leukemia cells (P388/ADM), which were resistant to multiple anticancer drugs. Quasipanaxatriol, 20(S)-protopanaxatriol, ginsenoside Rh2, and compound K greatly enhanced the cytotoxicity of the anti-cancer drugs in P388/ADM cells. The maximum increase in cytotoxicity was observed with 50 μM quasipanaxatriol; the resistance indices defined to be the ratios of the IC50 values for P388/ADM and P388 parental cells decreased significantly for both DAU and VBL. Hasegawa et al. hypothesized that reversal of DAU resistance in P388/ADM by quasipanaxatriol was due to the effective accumulation of the drugs mediated by the DAU-efflux blockage.

Gao et al., in Zhongguo Zhong Xi Yi Jie He Za Zhi, 19(1):17-9 (1999), investigated the potentiated effects of total saponins of Panax Ginseng (TSPG) on inhibition of leukemic progenitor cells by cytotoxic drugs in acute myelocytic leukemia. The investigators used bone-marrow cultures from the cells of 18 patients afflicted with acute myelocytic leukemia to assay the sensitivity of the leukemic cells to homoharringtonin (HHr), cytarabine (Ara), adriamycin (Adr) and etoposide (VP-16). In the presence of TSPG, they found that the inhibition rates of the leukemic cells treated with HHr, Ara, Adr and VP-16 were significantly higher than non-TSPG control (all P<0.01). From this data, Gao et al. concluded that TSPG could drive non-cycling leukemic progenitors to enter cell cycle, and thereby enhancing their susceptibility to cytotoxic drugs.

Keum et al., in Cancer Lett, 13;150(1):41-8 (2000), demonstrated that a methanol extract of heat-processed Panax ginseng C. A. Meyer could be used to scavenge superoxide generated by 12-O-tetradecanoylphorbol-13-acetate (TPA) in differentiated human promyelocytic leukemia (HL-60) cells. Keum et al. also showed, in Mutat. Res., 523-24:75-85 (2003), that Rg(3), a major ginsenoside derived from heat-processed ginseng, inhibited the TPA-induced activation of the eukaryotic transcription factor, NF-kappaB, in HL-60 cells.

Wang et al., in Zhongguo Zhong Xi Yi Jie He Za Zhi, 14(6):1089-95 (2006), tested the modulating effects of ginseng saponin, and other compounds, alone or in combination with cyclophosphamide (CTX) when these compounds were used to treat human erythroleukemic cell line K562 cells. Effects were assessed by measuring changes in telomerase activity of treated cells. The results showed that ginseng saponin or CTX could completely inhibit the telomerase activity of K562 cells at proper concentrations and exposure time. The inhibiting effects were enhanced when ginseng saponin was used with CTX. Telomerase activity decreased proportionally with the concentrations of each compound and length of time of exposure. Additionally, viability of K562 cells was decreased after co-culturing with ginseng and CTX, with increased levels of inhibition seen with increasing concentrations and exposure time.

Chui et al, in Oncol Rep. 16(6): 1313-6 (2006), tested the anti-leukemia potential of a combination regimen including crocodile egg extract, wild radix ginseng and natural Ganoderma lucidum (CGG extract) on acute myelogenous leukemia (AML) in vitro. The investigators tested the CGG extract's antiproliferative activity on the KG1a AML cell line and two freshly prepared bone marrow aspirate samples isolated from patients with de novo AML. Rats were tested in vivo with an excessive dose of CGG extract only to determine any development of acute toxicity. Chui et al. concluded that the CGG extract has growth inhibitory potential on KG1a cells and AML bone marrow samples in vitro. Additionally, their in vivo toxicity test revealed that no acute toxicity was observed after feeding the rats a high dosage of the CGG extract.

All of the aforementioned studies were limited by the fact that each study tested the effectiveness of saponin (ginsenoside) fraction of the ginseng plant under in vitro conditions. When in vivo conditions were implemented, it was only to test toxicity, not the effectiveness of the ginseng derived compound against a hematological malignancy. In one of the few studies that tested the effectiveness of ginseng derived components in vivo, Kim et al., in Immunopharmacol Immunotoxicol. 12(2):257-76 (1990), examined Panax ginseng for its immunomodulatory properties in mice. The main objective of this study, however, was to assay the effectiveness of ginseng derived components in inhibiting carcinogenesis, not to treat a hematological malignancy.

Kim et al. treated mice with cyclophosphamide to suppress their immune systems or with polyinosinic acid:polycytidylic acid (Poly I:C), an interferon inducer. Additionally, the mice were exposed to subchronic levels of Panax ginseng and challenged with transplanted syngeneic tumor cells. Following treatment, the researchers assayed the levels of multiple murine immune system components and determined that that Panax ginseng exposure stimulated basal natural killer (NK) cell activity in cyclophosphamide-immunosuppressed mice, but did not stimulate NK activity in Poly I:C treated mice. The investigators, additionally, found that other immunological parameters were not affected, including T and B cell responses. Although Panax ginseng stimulated increases in NK cell activity, this stimulation did not inhibit the growth of transplanted syngeneic tumor cells. Kim et al. did not address whether stimulation of NK cells would be effective against hematological malignancies, such as developing leukemias and lymphomas.

Although the above mentioned studies offer promising leads, an effective treatment for hematological malignancies is currently desired. For example, an estimated 35,070 new cases of leukemia will be diagnosed in the United States in 2006. Current treatments for leukemia are limited to chemotherapy, irradiation, stem cell transplantation, allogeneic bone marrow transplant, leukapheresis, etc. Chemotherapy is primarily used as a first line defense to trigger the elimination of leukemic cells from bone marrow samples, thereby causing remission. These drugs must often be combined to prevent drug resistance in leukemic cells and to stop the cancer from spreading to the central nervous system. However, the side-effects of chemotherapy are so devastating that many patients cannot and do not withstand the entire regimen prescribed. Despite the myriad of treatments available to combat the disease, approximately 22,280 deaths will be attributed to leukemia in 2006 in the United States, alone.

Thus, methods for treating hematological malignancies, such as leukemia and lymphoma are needed. Such a method should be able to effectively cure the hematological malignancy, yet still be tolerable to the patient, i.e., given in high doses for long periods of time, or even daily throughout life, free of debilitating side effects. Therefore, there is a need in the art for methods that are capable of triggering the patient's immune defenses in a manner that effectively eradicates malignant cells from the patient, while leaving the patient able to complete an entire treatment regimen without concomitant suffering from its side-effects.

### SUMMARY OF THE INVENTION

The present inventors have found that certain American ginseng extracts have immunoregulating properties. CVT-E002, PQ₂, PQ₂₂₃ and purified fractions from CVT-E002, PQ₂ and PQ₂₂₃, specifically stimulate the proliferation of NK and monocyte cells in the bone marrow and the spleen, which subsequently produces a reduction in the number of erythroleukemia cells found in the bone marrow and blood. These fractions may be used for the prevention or treatment of hematological malignancies.

Therefore, the present invention is directed to a Method of treating a condition characterized by a hematological malignancy comprising administering to the subject a condition treating effective amount of at least one ginseng fraction selected from the group consisting of CVT-E002, PQ₂, PQ₂₂₃ and purified fractions from CVT-E002, PQ2 and PQ₂₂₃.

The invention further includes a method of treating leukemia in a patient in need thereof, comprising administering to the patient a leukemia treating effective amount of at least one ginseng fraction selected from the group consisting of CVT-E002, PQ₂, PQ₂₂₃ and purified fractions from CVT-E002, PQ₂ and PQ₂₂₃.

The invention also includes a method of activating the proliferation of hemopoietic cells in a patient in need thereof, comprising administering to the patient a hemopoietic cell proliferating effective amount of at least one ginseng fraction selected from the group consisting of CVT-E002, PQ₂, PQ₂₂₃ and purified fractions from CVT-E002, PQ₂ and PQ₂₂₃.

The invention further includes a method of activating, in the bone marrow and the spleen, the proliferation of natural killer (NK) cells and monocytes in a patient in need thereof, comprising administering to the patient a natural killer (NK) and monocyte cell proliferating effective amount of at least one ginseng fraction selected from the group consisting of CVT-E002, PQ₂, PQ₂₂₃ and purified fractions from CVT-E002, PQ₂ and PQ₂₂₃.

### BRIEF DESCRIPTION OF THE DRAWINGS

FIG. 1 shows the effect of ginseng fraction CVT-E002 on hemopoietic and immune cell populations in the bone marrow of mice given 2 mg/day of CVT-E002.

FIG. 2 shows the effect of ginseng fraction CVT-E002 on hemopoietic and immune cell populations in the spleen of mice given 2 mg/day of CVT-E002.

FIG. 3 shows the effect of ginseng fraction CVT-E002 on hemopoietic and immune cell populations in the blood of mice given 2 mg/day of CVT-E002.

FIG. 4 shows the survival of mice given 2 mg/day of CVT-E002. Control: 10 mice; CVT-E002-treated: 15 mice.

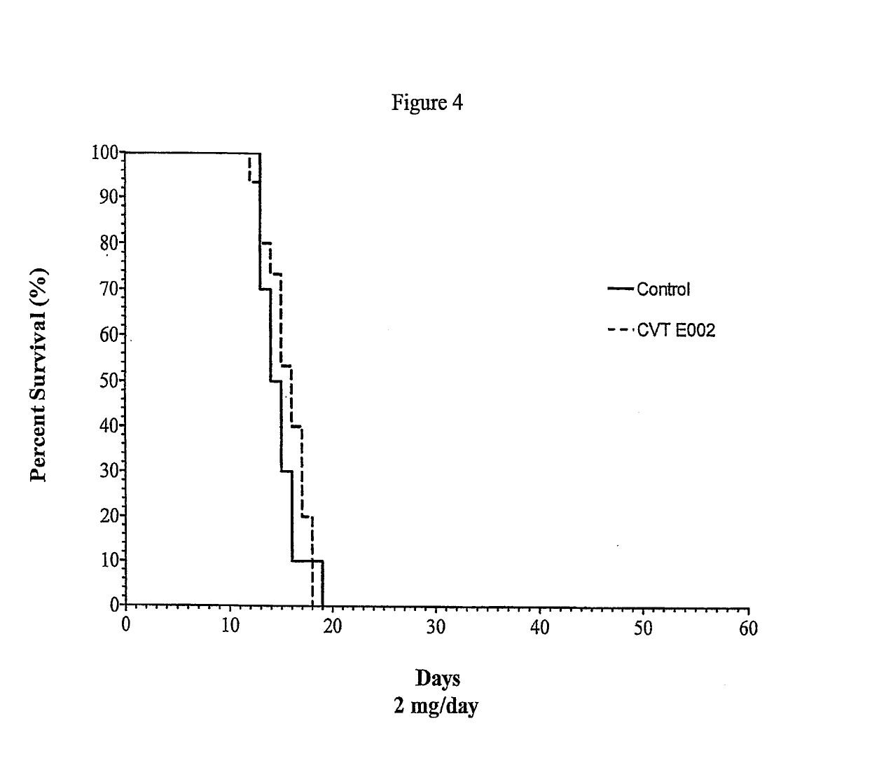

FIG. 5 shows the survival of mice given 40 mg/day of CVT-E002. Control: 10 mice; CVT-E002-treated: 4 identical groups of 10-11 mice each.

FIG. 6 shows the effect of ginseng fraction CVT-E002 on hemopoietic and immune cell populations in the bone marrow of mice given 40 mg/day of CVT-E002.

FIG. 7 shows the effect of ginseng fraction CVT-E002 on hemopoietic and immune cell populations in the spleen of mice given 40 mg/day of CVT-E002.

FIG. 8 shows the effect of ginseng fraction CVT-E002 on hemopoietic and immune cell populations in the blood of mice given 40 mg/day of CVT-E002.

FIG. 9 shows the survival of mice given 120 mg/day of CVT-E002. Control: 10 mice; CVT-E002-treated: 4 identical groups of 10-11 mice each.

### DETAILED DESCRIPTION OF THE INVENTION

The present invention is directed to a method of treating a condition characterized by a hematological malignancy comprising administering to the subject a condition treating effective amount of at least one ginseng fraction selected from the group consisting of CVT-E002, PQ₂, PQ₂₂₃ and purified fractions from CVT-E002, PQ₂ and PQ₂₂₃. Preferably, the condition characterized by a hematological malignancy is selected from the group consisting of abnormal proliferation of blood cells, a disease of the lymph nodes and multiple myeloma. Even more preferably, the abnormal proliferation of blood cells is selected from the group consisting of acute lymphocytic leukemia, acute myelogenous leukemia, chronic lymphocytic leukemia and chronic myelogenous leukemia.

The present invention further includes a method of treating leukemia in a patient in need thereof, comprising administering to the patient a leukemia treating effective amount of at least one ginseng fraction selected from the group consisting of CVT-E002, PQ₂, PQ₂₂₃ and purified fractions from CVT-E002, PQ₂ and PQ₂₂₃. Processes for making these ginseng fractions from a water soluble extract of the root portion of Panax quinquefolium have previously been described in U.S. Pat. Nos. 6,432,454; 7,067,160 and 7,186,423 and U.S. patent application Ser. No. 11/114,089 and 10/186,733 and are hereby incorporated by reference. Unlike the investigations described in the Background of the Invention, which focused on the toxicity and effectiveness of ginsenoside fractions, the present invention employs a non-ginsensoside containing polysaccharide extract of the ginseng plant. The processes for making the ginseng fractions of the present invention remove the ginsenoside fractions during the extraction process. Without being bound to any particular theory, the inventors believe that the polysaccharide extract effectuates immuno-surveillance against both developing tumors and cells infected with viruses that can cause leukemia.

The present invention also includes a method of activating the proliferation of hemopoietic cells in a patient in need thereof, comprising administering to the patient a hemopoietic cell proliferating effective amount of at least one ginseng fraction selected from the group consisting of CVT-E002, PQ₂, PQ₂₂₃ and purified fractions from CVT-E002, PQ₂ and PQ₂₂₃. Preferably, the hemopoietic cells activated are natural killer (NK) cells, precursor granuloid cells, mature granulocytes, erythroctyes and monocytes. Even more preferably, the proliferation of these cells occurs in the bone marrow, blood and/or the spleen.

Without being bound to any particular theory, the inventors believe that phytoceutical stimulation of the proliferation of NK cells and monocytes in the bone marrow facilitates immuno-surveillance against both developing tumors and cells infected with viruses that can cause leukemia. For example, Friend, murine and feline leukemia viruses are known to those of skill in the art to be etiologic agents in the animals in which they Infect. In humans, cases of Acute Myelogenous Leukemia can be associated with viral infections by either human immunodeficiency virus (HIV) or human T-lymphotropic virus (HTLV-1 and-2, which causes adult T-cell leukemia/lymphoma). In regards to non-viral leukemic tumorogenesis, cited factors include genetic predisposition and certain environmental conditions, e.g., chronic exposure to benzene, extraordinary exposure to ionizing irradiation, etc. Other factors and causes are yet to be discovered. Regardless of the cause of leukemia, however, the phytoceutical stimulation methods of the present invention are effective in treating the affliction by stimulating the proliferation of NK cells and monocytes in the bone marrow for subsequent immuno-surveillance functions against neoplastic cells in the bone marrow, blood and spleen.

The present invention is also effective against virally induced cancer. Without being bound to any particular theory, the inventors believe that phytoceutical stimulation is also effective against both developing tumors and cells infected with viruses that can cause cancer. In humans, it is currently estimated that 20-25% of human cancers are caused by viruses. For example, cases of gastric cancer, Burkitt's lymphoma, and Hodgkin's lymphoma are closely associated with Epstein-Barr viral infections. Prostate cancer cells have been found to be infected with XMRV retrovirus. Human papillomavirus (HPV) types 16 and 18 cause 70% of cervical cancer cases. Of patients afflicted with hepatocellular carcinoma (HCC), 20% are also afflicted with chronic viral hepatitis (hepatitis B or hepatitis C). Kaposi sarcoma-associated herpes virus is responsible for all forms of Kaposi sarcoma. Regardless of the virus that induces the cancer, the phytoceutical stimulation methods of the present invention are effective in treating the affliction by stimulating the proliferation of NK cells and monocytes in the bone marrow for subsequent immuno-surveillance functions against virally infected neoplastic cells.

Dosages of ginseng fractions in accordance with the invention depend upon the particular condition to be treated, as well as the age, sex and general health condition of the patient. However, suitable dosages may be found in the range 0.5 to 5000 mg/kg body weight. A preferable range for a suitable dosage of ginseng fractions is within the range of 1 to 4800 mg/kg body weight. Even more preferably, the suitable ginseng fraction dosage is within the range of about 3 to 1600 mg/kg body weight. The suitable dose should be administered in the range of 1 to 10 daily doses. Preferably, the suitable dose should be 1 to 5 daily doses. Even more preferably, the daily dose should be 2 to 4 daily doses. The ginseng fractions may be administered orally, via injection or infusion, topically, nasally, ocularly, vaginally or rectally.

The present invention encompasses uses of at least one ginseng fraction alone or in combination with another medicament. The present invention also encompasses uses of at least one ginseng fraction alone or in combination with a pharmaceutically acceptable carrier. Pharmaceutically acceptable carriers for use in the invention have previously been described in U.S. Pat. Nos. 6,432,454; 7,067,160 and 7,186,423 and U.S. patent application Ser. Nos. 11/114,089 and 10/186,733 and are hereby incorporated by reference. The ginseng fractions are especially suitable for co-administration with a chemotherapeutic agent or as a supplement to radiation therapy or stem cell transplantation, allogeneic bone marrow transplant, leukapheresis, etc.

The invention will now be further elucidated by the following Examples.

### EXAMPLE 1

### Effect of CVT-E002 on Hemopoietic and Immune Cell Populations in the Bone Marrow, Spleen and Blood of Mice given 2 mg/day of CVT-E002

Animals:

Eight to 9 week old male DBA/2 strain mice (Charles River Laboratories, St. Constant, QC, Canada) were housed upon arrival, one/cage and maintained under pathogen-free conditions (micro-isolator cages) in a temperature/humidity regulated facility with a 12 hour day/night cycle, in the Animal Care Facility of McGill University. The Facility is under continuous veterinary surveillance and strictly adheres to the regulations of the CCAC (Canadian Committee on Animal Care). Animals were provided water and food ad libidum, and remained undisturbed until 10 weeks old, the age of experiment initiation. Regular assessment of sentinel mice contained in the Facility consistently demonstrated the absence of all common mouse pathogens.

Tumor Cells—Maintenance and Administration:

Friend-virus-induced erythroleukemia cells (American Type Culture Collection, Manassas, Va., USA), were maintained in vitro at 37° C., 100% humidity and 5% CO₂, in Basal Eagle's Medium supplemented with 15% Cellect Gold™ Serum (MP Biomedicals, Solon, Ohio, USA), 4% essential amino acids, 2% non-essential amino acids, 1% L-glutamine (GIBCO, Invitrogen, Burlington, ON, Canada), 3% sodium bicarbonate (7.5% solution) and 1% HEPES (Sigma Aldrich, Oakville, ON, Canada), at a concentration of 2M. The pH was adjusted to 7.2 and the medium of the tumor cell stock was replenished 3 times/week. From this in vitro-maintained tumor cell stock, cells were extracted to initiate tumor-bearing hosts. Tumor cells were used during log phase growth when they showed an average viability of 89.4±0.64%. Each mouse was aseptically injected with 3×10⁶ viable tumor cells in 0.1 ml Phosphate Buffered Saline (PBS) at pH 7.2 via the lateral tail vein.

In Vivo Administration of CVT-E002:

CVT-E002, a proprietary extract of North American ginseng (Panax quinquefolius) comprised of unique polysaccharides (poly-furanosyl-pyranosyl-saccharides), of CV Technologies Inc., Edmonton, AB, Canada, was administered via the diet. Powdered extract of CVT-E002 was homogenized in finely ground standard Purina Laboratory mouse chow, (“Labchow,” Agribrands, Canada), the standard diet for all mice in the Facility. All feedings described below, began immediately after leukemia cell injection. All such leukemia-bearing mice were provided each morning (8:00-10:00 a.m.) with fresh ground chow with/without (control) the ginseng extract. Other mice (normal, non-leukemic) consumed regular untreated, pellet food. Each leukemic, experimental mouse was provided with 2 mg, 40 mg or 120 mg of CVT-E002 in 6 gm of ground chow/day, while leukemic, control mice consumed untreated ground chow only. Exhaustive previous studies in our laboratory have revealed that male mice of this strain and age regularly consume 6 gm of chow/day, virtually all of which is consumed during the dark phase of the 24 hour cycle.

Preparation of Free Cell Suspensions of Bone Marrow, Spleen and Blood:

Mice were killed by CO₂ asphyxiation at 10 days or 6 weeks after beginning CVT-E002 in the diet, as were corresponding control mice receiving untreated chow. Normal mice (above-mentioned) of the same strain, age and gender, were also assayed as described herewith. Single cell suspensions of the bone marrow and spleens were prepared by standard laboratory methods. Briefly, both femurs (bone marrow source) and the spleen were aseptically removed from each mouse and transferred to ice cold Minimal Essential Medium (MEM) (GIBCO Invitrogen, Burlington, ON, Canada), containing 10% heat-inactivated (56° C., 30 min) millipore-filtered Fetal Bovine Serum (FBS) (GIBCO, Invitrogen Burlington, ON, Canada). Spleens were pressed through a stainless steel screen mesh into medium, and bone marrow was removed from the femurs by repeated flushing of the contained cells with medium. Free cell suspensions from both organs were obtained by gentle repeated pipetting. The resulting suspensions were then layered for 7 min onto 1.5 ml Newborn Calf Serum (NCS) (GIBCO Invitrogen, Burlington, ON, Canada), to allow the sedimentation of any aggregates (non-cellular debris) into the pure NCS. The resulting aggregate-free supernatants were removed and centrifuged for 7 min (1100 rpm, 4° C.) and the resulting pellets were re-suspended in a fixed volume of fresh medium. The total number of nucleated cells was obtained by means of a hemocytometer (American Optical Co., Buffalo, N.Y., USA), and ‘the viability of the cells was simultaneously determined via the Trypan Blue dye exclusion method (0.04% dye in PBS of pH 7.2) (GIBCO Invitrogen, Burlington, ON, Canada). Bone marrow and spleen cell suspensions were adjusted to a final concentration of 40×10⁶ cells/ml.

Blood from every mouse (experimental, control, and normal) was transferred onto Superfrost Plus™ microscope slides (Fisher Scientific, Ottawa, ON, Canada), from a nick (via a sterile needle) in the lateral tail vein while the mouse was alive, and immediately prior to euthanizing for harvesting the bone marrow and spleen (above). Blood smears were then stained with MacNeal's tetrachrome hematologic stain (Sigma Aldrich, Oakville, ON, Canada) which permits the ready identification of several morphologically distinct cell types/lineages. After staining, the smears were cover-slipped and subsequently analyzed for 5 distinct cell types via light microscopy (×100). From counts of 1000 cells on each blood smear, the proportions of each cell type (mature granulocytes, granulocytic precursors, nucleated erythroid cells, lymphocytes, and monocytes) was obtained.

Immunolabelling of NK Cells:

ASGM-1 (asialogangliotetrasyliramide) is a surface molecule which is present on all mature and maturing NK cells. See Kasai et al., Eur. Jour. Immunol., 10: 175-180 (1980) and Beck et al., Transplantation, 33: 118-122 (1982). Although T lymphocyte blast cells also may possess it, these cells are not only rare but are easily distinguishable from NK cells both morphologically, by size and by our tetrachrome staining methods (above).

The NK cell immunolabelling method described presently is well established in our laboratory. See Currier et al., Jour. Altern. Comp. Med., 7(3): 241-251(2001), Currier, et al., Jour. Alter. Comp. Med., 8(1): 49-58(2002), Brousseau, et al., Biogeron, 6:157-163, (2005), Miller, et al., Nat. Immun., 11:78-91 (1992), Dussault, et al., Nat. Immun., 12: 55-78 (1993), Dussault, et al., Nat. Immun., 14: 35-43 (1995) and Currier, et al., Exp. Geront., 35: 627-639 (2000). Using 96 multi-well plates (Sarstedt, Montreal, QC, Canada), 100 μl of the bone marrow and spleen cell suspensions (above) were incubated with 100 μl of primary antibody, rabbit anti-ASGM-I (Wako Pure Chemicals, Dallas, Tex., USA) at a dilution of 1:40 in medium for 30 min on ice. After incubation, the cells were centrifuged for 7 min (1100 rpm, 4° C.), followed by two consecutive washes in 100 μl of medium and centrifuging as above. After the final wash, the pellets were re-suspended and incubated with 100 μl of the secondary biotinylated antibody, anti-rabbit IgG (Sigma Aldrich, Oakville, ON, Canada) at a concentration of 1:100 in medium for 30 min on ice. Cell suspensions were again centrifuged and washed twice as above before being re-suspended in 4.5 ml of cytospotting medium (0.009% NaCl, 0.001% EDTA and 0.05% bovine serum albumin (BSA) in distilled water (pH 7.4) (Sigma Aldrich, Oakville, ON, Canada). The cells were then cytocentrifuged (5 min, 1000×g) onto Superfrost Plus™ microscope slides (Fisher Scientific, Ottawa, ON, Canada), and rapidly air-dried to avoid cell shrinkage. The slides were then fixed in pure methanol for 30 min on ice and rehydrated progressively (25%, 50%, 75% and 100%) for 5 min with PBS pH 7.2, bathed for 10 min in a 3% hydrogen peroxide solution to block endogenous peroxidase activity (Fisher Scientific, Ottawa, ON, Canada). The slides were washed for 10 min in PBS and then incubated with 100 μl of avidin-biotin horseradish peroxidase complex (ABC) solution (Dako Diagnostics, Mississauga, ON, Canada) for 45 min in a fully humidified chamber. Next, the slides were washed as above in PBS to removed any residual ABC solution before being immersed in a 3-3′diaminobenzidine solution (DAB) (0.125 g DAB, 66.6 μl of 30% H₂O₂ in 250 ml PBS at pH 7.6) for 13 min followed by two consecutive washes in PBS. The slides containing the cytospots were blotted dry and subsequently stained with MacNeal's tetrachrome hematologic stain, and cover-slipped. Thus, the double-staining (immunolabelling and tetrachrome staining) method permits ready identification of the 5 cell types mentioned above, including NK cells, the latter readily segregated and distinguished by means of their ASGM-1 surface marker from all other lymphocytes, i.e., mature and maturing T and B lymphocytes, from which they are otherwise morphologically indistinguishable.

Differential Analysis of Hemopoeitic and Immune Cells in the Bone Marrow and Spleen:

In both the bone marrow and spleen, mature granulocytes, granulocytic precursors (immature granuloid cells), nucleated erythroid cells, NK lymphocytes, non-NK lymphocytes (i.e., T and B, and monocytes), were identified, using light microscopy at ×100. The differential counts were obtained via this method, from 1000 spleen and blood cells/cytospot/mouse, and 2000 bone marrow cells/cytospot/mouse for every experimental (CVT-E002-containing chow), control (untreated chow), and normal mouse. For each organ, and for each mouse, the percentages for each cell group (above) were recorded. The absolute numbers of NK cells and their accessory cells, the monocytes, were then obtained by converting these percentage values per organ, via the known total cellularity of that organ, recorded from the hemocytometer at the time of animal death.

Statistical Analysis:

The two-tailed Student t-test was used to compare the differences between the means of the experimental (CVT-E002-treated) and corresponding control groups. Values of p<0.05 were considered statistically significant. All specific p values are recorded on the histograms and within the tables.

Results:

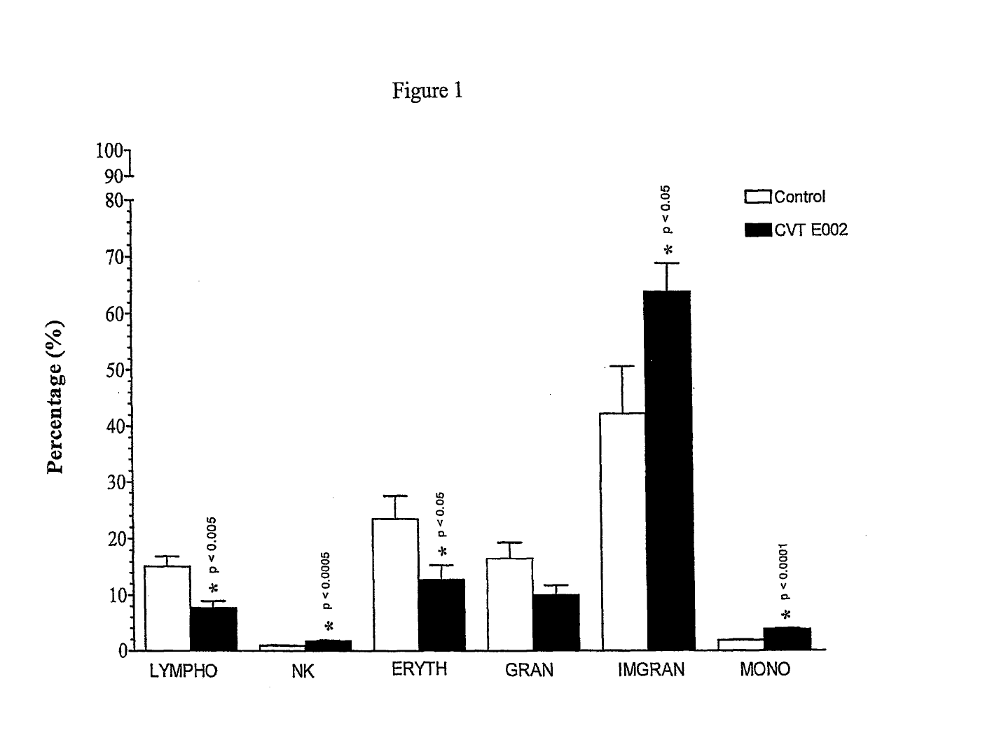

For FIGS. 1-3 and 6-9, the following abbreviations are used: LYMPHO=lymphocytes, including T and B cells; NK=natural killer cells; RBC=red blood cell proliferating precursors; GRAN=mature (functional) granulocytes; IMGRAN=immature granulocytes, i.e., proliferating precursors; MONO=monocytes. N=8 samples (mice)/cell type; *=statistically significant p values vs. control assessed by means of the two-tailed student “t” test. Levels of significance are indicated.

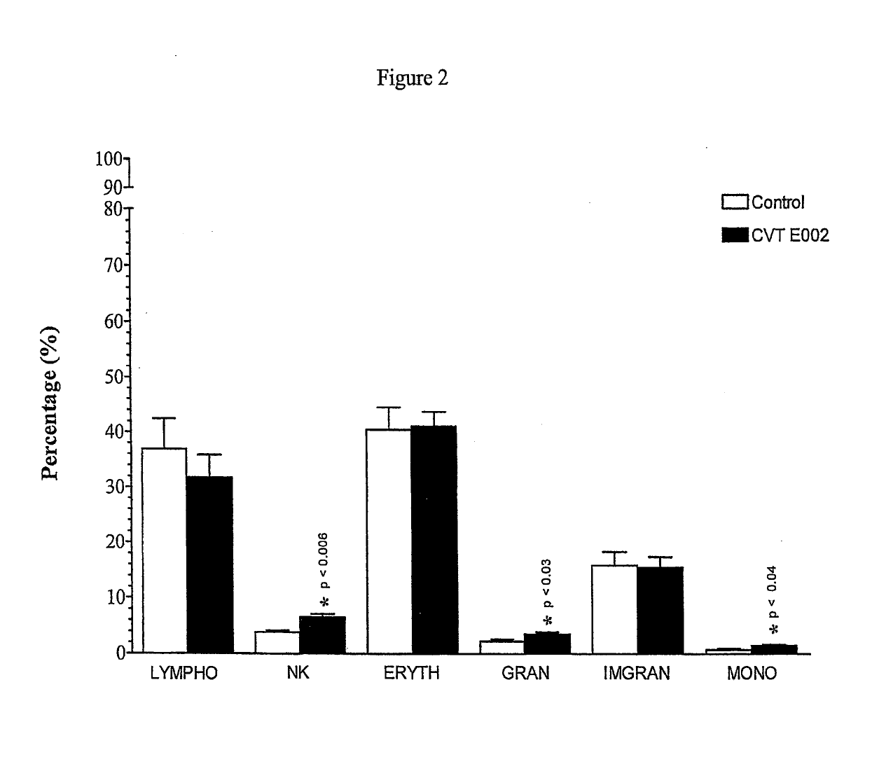

FIG. 1 demonstrates the effect of 2 mg/day of CVT-E002 on the central generating site of all hemopoietic and immune cells, i.e., the bone marrow. The data Indicate that there has been a profound influence of CVT-E002 on several of the cell lineages in that organ. Particularly important is the observation that NK cells had doubled their levels in the CVT-E002-consuming group of leukemic mice vs. leukemic mice on untreated diet. A significant augmentation was also found with NK cells in the spleens of these animals (FIG. 2). These relative values, i.e., percentages (FIGS. 1, 2), when converted to absolute numbers, indicate that CVT-E002 has indeed produced augmentation in the population size of these vital anti-tumor cells (see Table 1 below).

TABLE 1
Absolute numbers of NK cells and Monocytes
in the Bone Marrow and Spleen
10 days post leukemia injection of mice treated
with daily dietary CVT-E002 at 2 mg/day

| | | | | MONO- |
|---|---|---|---|---|
| | | NK CELLS | | CYTES |
| | NK CELLS | CVT-E002 | MONOCYTES | CVT-E002 |
| | Control (×10⁶) | (×10⁶) | Control (×10⁶) | (×10⁶) |
| BONE | 0.11 ± 0.02 | 0.20 ± 0.06 | 0.20 ± 0.04 | 0.42 ± 0.09 |
| MARROW | N = 8 | N = 8 | N = 8 | N = 8 |
| SPLEEN | 11.46 ± 1.49 | 21.79 ± 2.83 | 2.82 ± 1.04 | 5.38 ± 1.05 |
| | N = 9 | N = 8 | N = 8 | N = 8 |

The necessary accessory cells for NK cells are the monocytes, and in the presence of 2 mg/day of CVT-E002, precisely the same phenomenon has occurred as observed for NK cells, in both the bone marrow and the spleens of these leukemic animals. That is, the proportions and absolute numbers of monocytes are elevated in the presence of CVT-E002 (FIGS. 1, 2; Table 1). Thus, in these leukemic mice, for both the tumor cytolytic NK cells and their monocyte helpers, CVT-E002 has had a very positive effect.

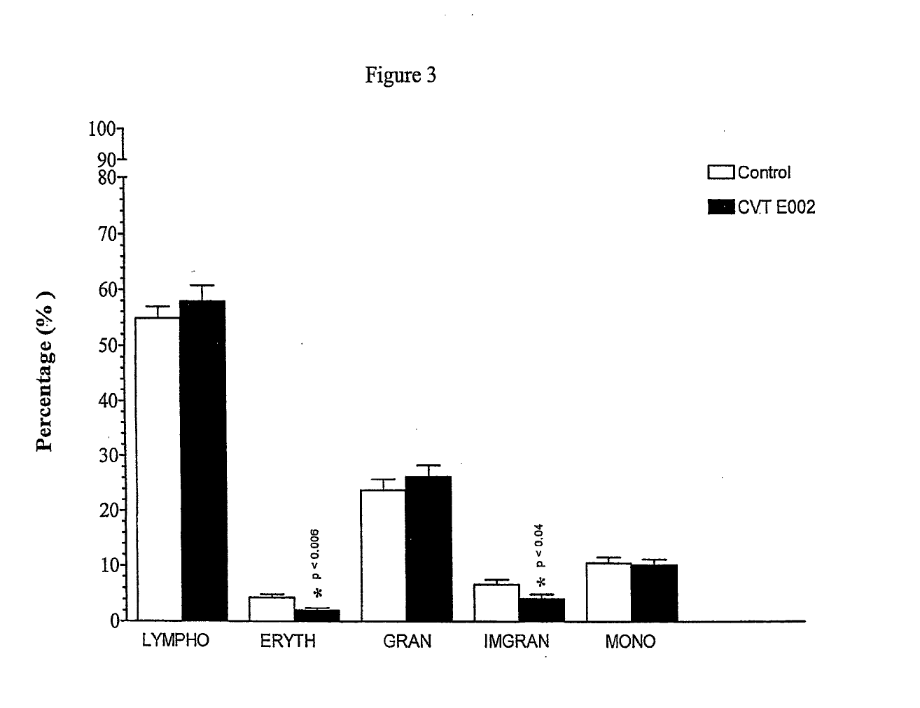

The next population of cells to come under the influence of CVT-E002 is that of the nucleated red blood cells. The leukemia under assay in this study is an erythroleukemia, and consequently, we have found that the vast majority of these nucleated red blood cells were blasts belonging to the tumor. CVT-E002 has significantly reduced the numbers of erythroid blasts in the bone marrow relative to control (FIG. 1) and the same phenomenon is observed in the blood, the only exit for all cells born in the bone marrow (FIG. 3).

The inter-organ (bone marrow→blood→spleen) population dynamics of the lymphocytes (T, B cells) in these leukemic mice, with and without 2 mg/day of CVT-E002, is shown in FIGS. 1-3. The bone marrow is the only generation site of all primary, virgin lymphocytes of the “B” lineage, the mature progeny of which are then disseminated via the blood to the spleen and to the numerous lymph nodes throughout the body. In the presence of CVT-E002, the B lymphocyte levels in the bone marrow have fallen significantly (FIG. 1). The total lymphocyte levels in the spleen (FIG. 2) are slightly reduced in the presence of CVT-E002. However, since the spleen also contains very large numbers of T lymphocytes (not of bone marrow origin), the slight reduction in the total lymphocyte levels is most probably mediated by the reduction in bone marrow-derived B lymphocytes. There has, correspondingly, been no change in the proportions of lymphocytes observed in the blood (FIG. 3). As usual, any observation concerning lymphocyte levels in the blood will be confounded by the fact that the blood is the highway along which all lymphocytes (T, B, NK) must travel to and from, among and between, the main hemopoietic and immune sites, i.e., bone marrow, spleen and several hundred peripheral lymph nodes.

With respect to cells of the granulocyte lineage, FIG. 1 demonstrates that in the bone marrow, immature, proliferating precursor granuloid cells are approximately one-third more prominent in the bone marrow under the influence of daily dietary CVT-E002 vs. control, untreated diet, contrasting with a one-third reduction (vs. control, untreated diet) in the levels of their mature progeny in that organ (FIG. 1). In the spleen, however, CVT-E002 has instigated an elevation in the levels of mature granulocytes (FIG. 2), vs. untreated, leukemic controls.

FIG. 3 indicates a significant reduction in the proportion of immature granulocytes in the blood of CVT-E002-consuming mice, vs. control, although, as above, this may not be significant in any functional sense since the blood is only a connecting route between and among many hemopoietic and immune cell-containing organs.

### EXAMPLE 2

The procedure of Example 1 is repeated, but in place of CVT-E002, an equal amount of PQ₂ is used. Results similar to those found in Example 1 are obtained.

### EXAMPLE 3

The procedure of Example 1 is repeated, but in place of CVT-E002, an equal amount of PQ₂₂₃ is used. Results similar to those found in Example 1 are obtained.

### EXAMPLE 4

### Effect of CVT-E002 on Hemopoietic and Immune Cell Populations in the Bone Marrow, Spleen and Blood of Mice given 40 mg/day of CVT-E002

The administration of tumor cells and CVT-E002 to mice, preparation of free cell suspensions of bone marrow, spleen and blood, immunolabelling of NK cells, differential analysis of hemopoeitic and immune cells in the bone marrow and spleen, and statistical analysis was in accordance with the materials and methods described in Example 1.

Results:

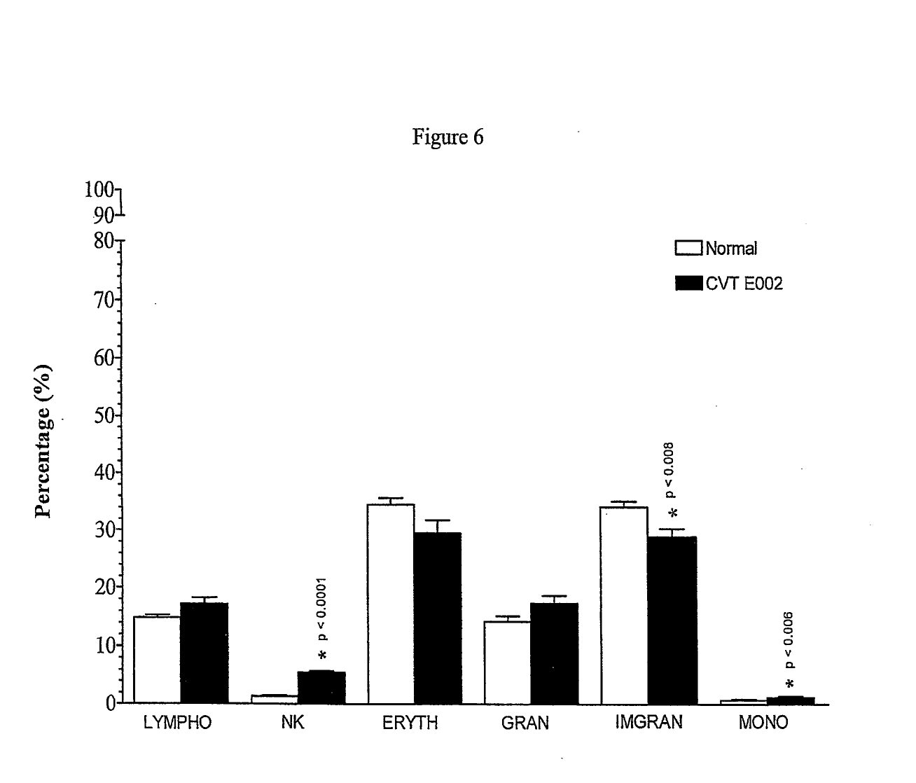

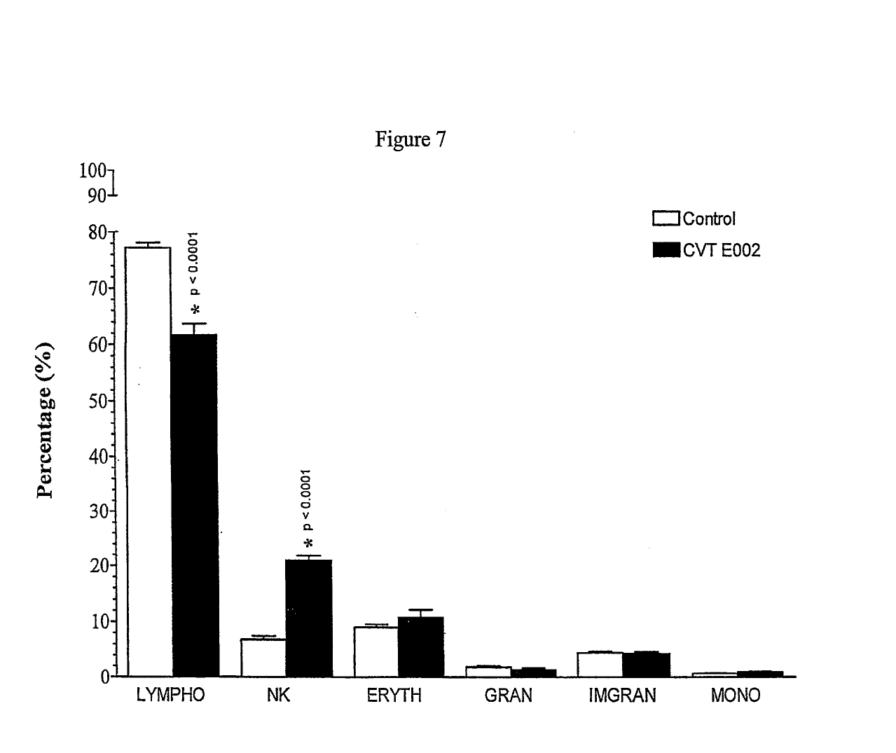

The hemopoietic and immune cell data recorded for leukemic mice receiving 40 mg/day CVT-E002 revealed that there were significant elevations in the proportions of NK cells and monocytes in the bone marrow (FIG. 6) and the spleen (FIG. 7) in leukemic, CVT-E002-treated mice at 6 weeks from tumor onset, vs. healthy, normal mice of matched strain, gender and age. For FIGS. 6-8, because no “control” (leukemia without treatment) mice lived until 6 weeks, the experimental values are compared to normal mice of the same strain, age and gender. That these proportions reflect absolute increases in the numbers of these cells in these organs is shown in Table 2. See below.

TABLE 2
Absolute numbers of NK cells and Monocytes
in the Bone Marrow and Spleen
Normal untreated mice and Leukemic mice treated
for 6 weeks with dietary CVT-E002 at 40 mg/day

| | | | | MONO- |
|---|---|---|---|---|
| | NK CELLS | NK CELLS | | CYTES |
| | Control | CVT-E002 | MONOCYTES | CVT-E002 |
| | (×10⁶) | (×10⁶) | Control (×10⁶) | (×10⁶) |
| | Mean ± SEM | Mean ± SEM | Mean ± SEM | Mean ± SEM |
| BONE | 0.32 ± 0.13 | 1.46 ± 0.07 \textcircled{1} | 0.19 ± 0.02 | 0.36 ± 0.04 \textcircled{3} |
| MARROW | | | | |
| SPLEEN | 6.44 ± 0.61 | 24.79 ± 1.82 \textcircled{2} | 0.70 ± 0.96 | 1.23 ± 0.20 \textcircled{4} |

\textcircled{1} p < 0.0001,
\textcircled{2} p < 0.005,
\textcircled{3} p < 0.0001,
\textcircled{4} p < 0.04

The proportions of all other hemopoietic and immune cells in the bone marrow and spleen were either at, or close to, normal levels for mice of the same age and gender (FIGS. 6, 7). Normal mice were used as a barometer to assess if and when the various hemopoietic and immune cell lineages returned to normal in leukemic mice treated with 40 mg/day of CVT-E002. Moreover, normal mice were used for comparison because all control, leukemic mice, i.e., those not receiving dietary CVT-E002, had died between day 15-19 of the test period.

A pivotal observation in both the spleen and the bone marrow or these originally leukemic mice, at 6 weeks after receiving 40 mg/day of CVT-E002, is that fact that the numbers of nucleated erythroid cells have returned to levels comparable to those of normal mice (FIGS. 6, 7), suggesting the presence of few if any remaining erythroleukemic cells, the latter being indistinguishable from endogenous, nucleated erythroid cells.

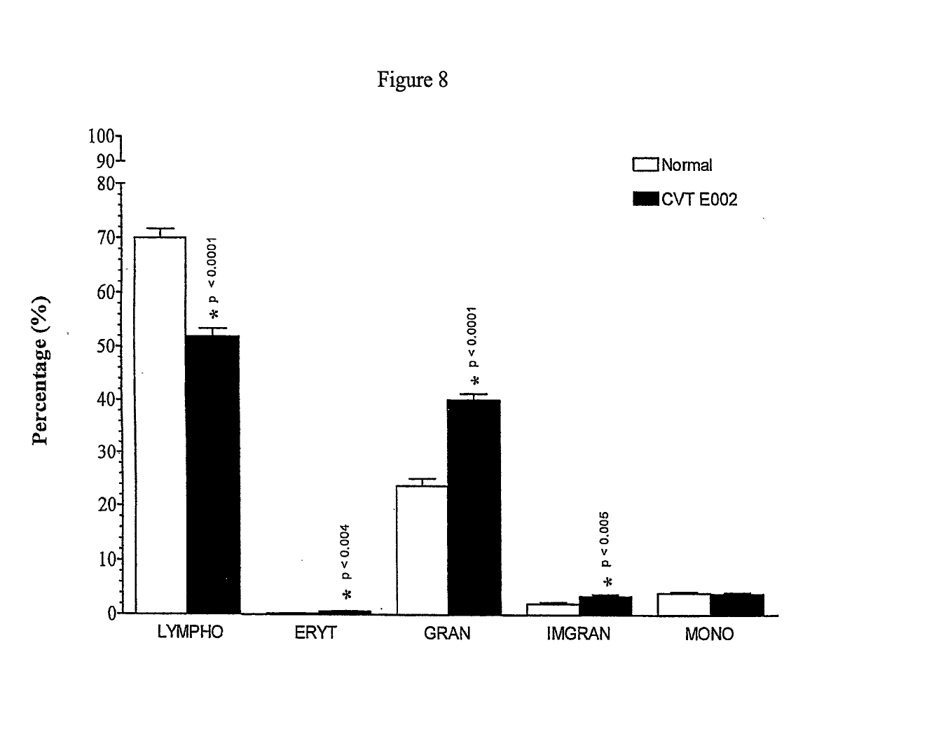

At the 6 week interval, the relative proportions of the various cell lineages were also recorded for the blood from both CVT-E002-treated and normal mice of corresponding strain, age and gender (FIG. 8). The lower proportions of lymphocytes in the blood of CVT-E002-treated mice, may simply reflect the fact that within this group are the NK cells and the proportions and absolute numbers of these cells are significantly higher in both the bone marrow and spleen (Table 2) of CVT-E002-treated leukemic mice vs. those of normal, untreated mice, resulting in fewer NK cells in transit in the blood. The higher proportions of immature and mature granulocytes in the blood would reflect their higher numbers in their bone marrow generating site, given that the blood circulation is the only route out of the bone marrow. Finally, the proportions of nucleated erythroid cells seen in this organ, in 6 week old, CVT-E002-treated, leukemic mice, although statistically elevated (FIG. 8), are very low (0.57±0.09%), and much closer to the levels in normal mice (0.17±0.07%) than they are to untreated, control leukemic mice (4.27±0.54%: FIG. 3). Thus, FIGS. 6, 7, 8 all reveal a return of the hemopoletic and immune cell values to normal levels, with the exception of the anti-tumor NK cells and monocytes, whose numbers remain beneficially elevated with sustained CVT-E002 administration.

### EXAMPLE 4

The procedure of Example 4 is repeated, but in place of CVT-E002, an equal amount of PQ₂ is used. Results similar to ‘those found in Example 4 are obtained.

### EXAMPLE 5

The procedure of Example 4 is repeated, but in place of CVT-E002, an equal amount of PQ₂₂₃ is used. Results similar to those found in Example 4 are obtained.

### EXAMPLE 6

### Effect of CVT-E002 on Mice Survival when Given 2, 40 and 120 mg/day of CVT-E002

Assessment of CVT-E002 Mediated Survival:

Groups of leukemic mice, fed daily with 2 mg, 40 mg or 120 mg CVT-E002, as well as a group of control leukemic mice consuming untreated chow, were left unmanipulated, to assess the influence of CVT-E002 treatment on life span. Kaplan-Meier Survival Analysis software was applied to assess the significance of CVT-E002, vs. no treatment, on life span of the leukemic mice.

Results:

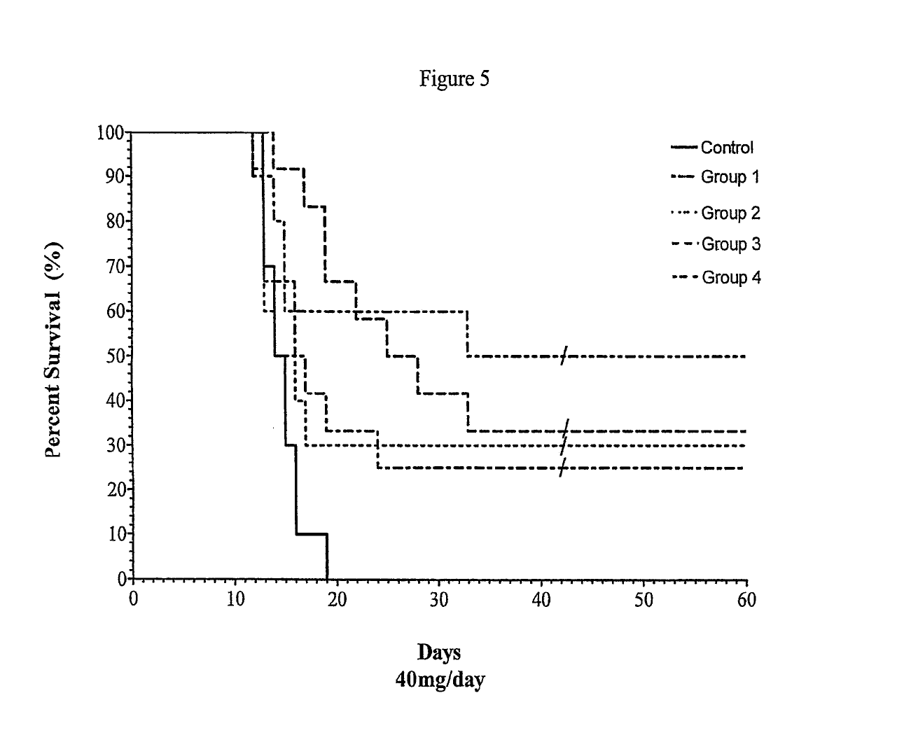

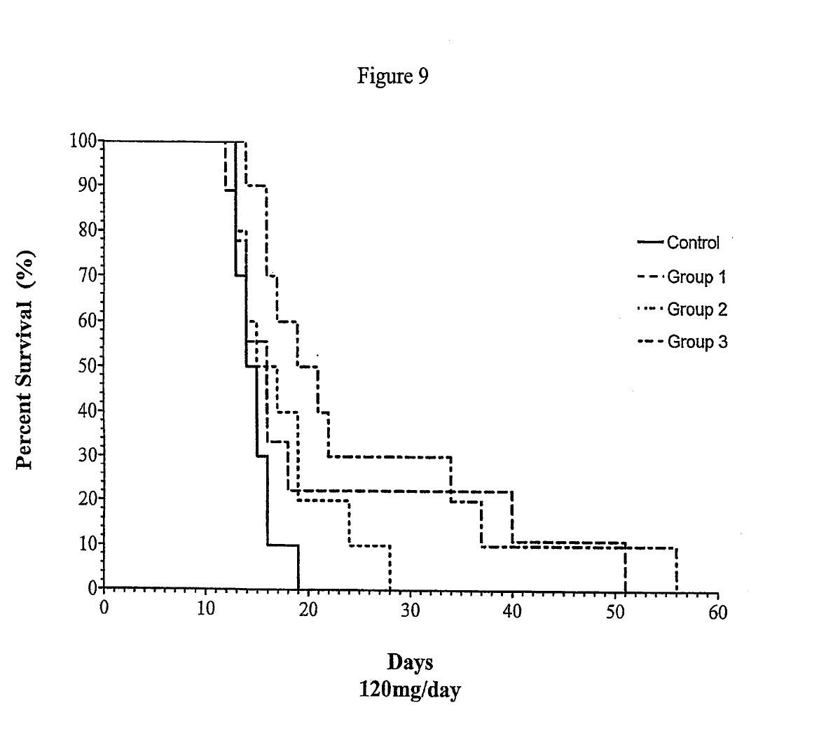

When increasing the daily dose of CVT-E002 in leukemic mice to 120 mg/day, an improvement in survival was observed in all groups tested (FIG. 9), however, the survival enhancement was considerably less impressive than that seen with 40 mg/day (FIG. 5). In fact, no leukemic mice treated with 120 mg/day, lived up to 6 weeks after leukemia onset (FIG. 9), indicating the significance of dosage in establishing therapeutic levels of CVT-E002.

In summary, the results of the present study show that the ginseng fractions of the invention significantly stimulated non-specific immunity in leukemic mice, and significantly extended the life span of leukemia-afflicted hosts. Moreover, specific changes imbued by CVT-E002 upon other hemopoietic and immune cells in the 3 key sites which contain such elements, i.e., the bone marrow (generating site of all hemopoietic/immune cells), the blood highway, and the spleen, the site to which all these cells ultimately transit, or within which they become functional residents. Significantly elevated levels were found in the absolute numbers of NK cells and monocytes, mediators of the first line of defense in leukemia combat, in both the bone marrow and the spleen of CVT-E002-treated leukemic mice. Concomitant with these observations is the finding of a significant reduction of erythroleukemia cells in the bone marrow of CVT-E002-fed leukemic mice. This study has shown (i) that approximately one-third to one-half of leukemic mice administered this agent went on to achieve a potentially normal life span, and (ii) that dosage is critical in producing these ameliorative effects. This pre-clinical study has both direct and immediate applicability to the human condition since the botanical used is already commercially available, in widespread use, inexpensive, and without debilitating side effects. Thus, CVT-E002 is a powerful new tool which can be added to, or even replace, existing arsenals for combating hematological malignancies such as leukemia and lymphomas, and other types of tumors, including tumors caused by viruses, as well.

### EXAMPLE 7

The procedure of Example 6 is repeated, but in place of CVT-E002, an equal amount of PQ₂ is used. Results similar to those found in Example 6 are obtained.

### EXAMPLE 8

The procedure of Example 6 is repeated, but in place of CVT-E002, an equal amount of PQ₂₂₃ is used. Results similar to those found in Example 6 are obtained.

1. A method of treating leukemia in a patient in need thereof, wherein said method comprises administering to said patient an effective amount of at least one ginseng fraction selected from the group consisting of CVT-E002, PQ₂, and PQ₂₂₃.

2. A method of treating a hematological malignancy, wherein said method comprises administering to the subject an effective amount of at least one ginseng fraction selected from the group consisting of CVT-E002, PQ₂, and PQ₂₂₃.

3. The method of claim 2, wherein the hematological malignancy is selected from the group consisting of an abnormal proliferation of blood cells, a disease of the lymph nodes and multiple myeloma.

4. The method of claim 3, wherein the blood cells are leukocytes and/or erythrocyte precursors.

5. The method of claim 3, wherein the abnormal proliferation of blood cells is selected from the group consisting of acute lymphocytic leukemia, acute myelogenous leukemia, chronic lymphocytic leukemia, chronic myelogenous leukemia and hairy cell leukemia.

6. The method of claim 5, wherein said acute myelogenous leukemia is erythroleukemia.

7. The method of claim 1, wherein the leukemia is a virally induced leukemia.

8. The method of claim 1, wherein the leukemia is erythroleukemia.

9. The method of claim 1, wherein the at least one ginseng fraction comprises CVT-E002.

10. The method of claim 1, wherein the at least one ginseng fraction is capable of activating proliferation of hemopoietic cells in the patient.

11. The method of claim 10, wherein the hemopoietic cells are selected from the group consisting of natural killer (NK) cells, precursor granuloid cells, mature granulocytes, erythrocytes and monocytes.

12. The method of claim 10, wherein said proliferation occurs in the bone marrow, blood and/or the spleen.

13. The method of claim 2, wherein the hematological malignancy is a virally induced hematological malignancy.

14. The method of claim 2, wherein the hematological malignancy is a leukemia.

15. The method of claim 2, wherein the at least one ginseng fraction comprises CVT-E002.

16. The method of claim 2, wherein the at least one ginseng fraction is capable of activating proliferation of hemopoietic cells in the patient.

17. The method of claim 16, wherein the hemopoietic cells are selected from the group consisting of natural killer (NK) cells, precursor granuloid cells, mature granulocytes, erythrocytes and monocytes.

18. The method of claim 16, wherein said proliferation occurs in the bone marrow, blood and/or the spleen.

19. A method of treating erythroleukemia in a subject comprising administering to the subject an effective amount of at least one ginseng fraction selected from the group consisting of CVT-E002, PQ₂ and PQ₂₂₃.
